(19) United States
(12) Patent Application Publication
(10) Pub. No.: US 2020/0171475 A1
(43) Pub. Date: Jun. 4, 2020

(54) DEHYDROCYCLISATION CATALYST FOR HYDROCARBONS

(71) Applicant: King Abdullah University of Science and Technology, Thuwal (SA)

(72) Inventors: Jean Marie BASSET, Thuwal (SA); Walid AL MAKSOUD, Thuwal (SA); Lieven GEVERS, Thuwal (SA); Jullian VITTENET, Thuwal (SA)

(21) Appl. No.: 16/639,321

(22) PCT Filed: Aug. 16, 2018
(86) PCT No.: PCT/IB2018/056185

§ 371 Date: Feb. 14, 2020

Related U.S. Application Data
(60) Provisional application No. 62/546,702, filed on Aug. 17, 2017.

(51) Int. Cl. B01J 31/16; B01J 29/40; B01J 37/02; B01J 37/08; C07C 2/76

(52) U.S. Cl. CPC B01J 31/1633; B01J 29/405; B01J 37/0209; C07C 2531/22; B01J 37/08; C07C 2/76; C07C 2529/40; B01J 37/0207

(57) ABSTRACT

Embodiments of the present disclosure describe a catalyst for dehydrocyclisation of hydrocarbons comprising a suitable support and an organometallic complex or a coordination compound including at least a dehydrogenation metal, wherein the dehydrogenation metal of the organometallic complex or coordination compound is grafted to a selected site of the suitable support. Embodiments of the present disclosure further describe a method of preparing a dehydrocyclisation catalyst for hydrocarbons comprising grafting a dehydrogenation metal of an organometallic complex or coordination compound to a selected site of a suitable support to form the dehydrocyclisation catalyst. Another embodiment of the present disclosure is a method of dehydrocyclisation of hydrocarbons comprising contacting a hydrocarbon with a dehydrocyclisation catalyst to convert the hydrocarbon to an aromatic compound, wherein the dehydrocyclisation catalyst includes a dehydrogenation metal grafted to a selected site of a suitable support.

### BACKGROUND

As a considerable amount of light alkanes (e.g., propane and butane) are included in natural gas and formed as by-products in petroleum refineries, an economical need to upgrade these alkanes exists. The dehydrocyclisation of light alkanes, such as ethane, propane, and n-butane, is an important catalytic reaction, both industrially and catalytically, as it produces valuable aromatic chemicals, such as benzene, toluene, and xylenes. In general, the dehydrocyclisation of lower paraffinic hydrocarbons for the production of aromatic compounds includes four steps. The first step includes dehydrogenation using a Lewis acid. The second step includes oligomerization to, for example, C₆ to C₉ olefins using a Brønsted acid. The third step includes cyclisation using the Brønsted acid. The fourth step includes dehydrogenation of cyclic hydrocarbons using the Lewis acid to produce the aromatic compounds.

Conventional dehydrocyclisation processes use gallium or zinc deposited on ZSM-5 as a support due to its high selectivity for benzene, toluene, and xylene. The presence of the gallium species inside the molecular sieve is shown to provide the Lewis acidity necessary for dehydrogenation. In addition, other metals have been used, such as zinc, nickel, zirconium, iron, molybdenum, and silver, to modify the molecular sieves via conventional methods, such as wetness impregnation, ion exchange, and solid state synthesis. However, these processes use, and methods produce, catalysts with low stability and rapid deactivation due to coking inside the micropores of the catalyst and deposition on the catalyst surface. In addition, these conventional catalysts also exhibit low conversions, yields, and selectivities.

Accordingly, it would be desirable to provide a highly stable dehydrocyclisation catalyst with enhanced yields, conversions, and selectivities.

### SUMMARY

In general, embodiments of the present disclosure describe dehydrocyclisation catalysts, methods of preparing dehydrocyclisation catalysts, and methods of dehydrocyclisation of hydrocarbons to aromatics.

Accordingly, embodiments of the present disclosure describe a catalyst for dehydrocyclisation of hydrocarbons comprising a suitable support and an organometallic complex or a coordination compound including at least a dehydrogenation metal, wherein the dehydrogenation metal of the organometallic complex or coordination compound is grafted to a selected site of the suitable support.

Embodiments of the present disclosure further describe a method of preparing a dehydrocyclisation catalyst for hydrocarbons comprising grafting a dehydrogenation metal provided by an organometallic complex or a coordination compound to a selected site of a suitable support to form the dehydrocyclisation catalyst.

Another embodiment of the present disclosure is a method of dehydrocyclisation of hydrocarbons comprising contacting a hydrocarbon with a dehydrocyclisation catalyst to convert the hydrocarbon to an aromatic compound, wherein the dehydrocyclisation catalyst includes a dehydrogenation metal grafted to a selected site of a suitable support.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1A is a flowchart of a method 100A of preparing a dehydrocyclisation catalyst for hydrocarbons, according to one or more embodiments of the present disclosure.

FIG. 1B is a flowchart of a method 100B of preparing a dehydrocyclisation catalyst for hydrocarbons, according to one or more embodiments of the present disclosure.

FIG. 2 is a flowchart of a method 200 of dehydrocyclisation of hydrocarbons, according to one or more embodiments of the present disclosure.

FIG. 3 is a graphical view illustrating propane conversion over Parent-ZSM-5 (blue), [≡Si—O—ZnCH₃] (grey), [≡Si—O—TiNp₃] (black), and Ga@ZSM-5 (green); (reaction condition: 150 mg, T=550° C.; flow of the propane feed in reactor=20 mL/min; flow of N₂=2 mL/min), 12 h, according to one or more embodiments of the present disclosure.

FIGS. 4A-4B are graphical views illustrating (A) average selectivity of different products obtained during the reaction of propane over Parent-ZSM-5 (blue), [≡Si—O—ZnCH₃] (grey), [≡Si—O—TiNp₃] (black), and Ga@ZSM-5 (green); and (B) average yield of different products obtained during the reaction of propane over Parent-ZSM-5 (blue), [≡Si—O—ZnCH₃] (grey), [≡Si—O—TiNp₃] (black) and Ga@ZSM-5 (Green); (Reaction condition: 150 mg, T=550° C., Flow of the propane feed in reactor=20 mL/min, Flow of N₂=2 mL/min), 12 h), according to one or more embodiments of the present disclosure.

FIG. 5 is a graphical view illustrating conversion of propane in PID reactor (3 days) over Parent-ZSM-5 (blue), [≡Si—O—TiNp₃] (black), and Ga@ZSM-5 (green); (Reaction condition: 150 mg, T=550° C., Flow of the propane feed in reactor=20 mL/min, Flow of N₂=2 mL/min), 3 days), according to one or more embodiments of the present disclosure.

FIGS. 6A-6B is a graphical view illustrating (A) selectivity of Aromatics (benzene, toluene, para-xylene) during the reaction of propane over Parent-ZSM-5 (blue), [≡Si—O—TiNp₃] (black) and Ga@ZSM-5 (green); and (B) yield of Aromatics (benzene, toluene, para-xylene) during the reaction of propane over Parent-ZSM-5 (blue), [≡Si—O—TiNp₃] (black) and Ga@ZSM-5 (green) (Reaction condition: 150 mg, T=550° C., Flow of the propane feed in reactor=20 mL/min, Flow of N₂=2 mL/min), 3 days), according to one or more embodiments of the present disclosure.

FIGS. 7A-7B is a graphical view illustrating (A) selectivity of C₃ (e.g., Propylene) obtained during the reaction of propane over Parent-ZSM-5 (blue), [≡Si—O—TiNp₃] (black) and Ga@ZSM-5 (green); and (B) yield of C₃ (e.g., propylene) obtained during the reaction of propane over Parent-ZSM-5 (blue) and Ga@ZSM-5 (green) (Reaction condition: 150 mg, T=550° C., Flow of the propane feed in reactor=20 mL/min, Flow of N₂=2 mL/min), 3 days), according to one or more embodiments of the present disclosure.

### DETAILED DESCRIPTION

The invention of the present disclosure relates to catalysts for the dehydrocyclisation of hydrocarbons. In particular, the invention of the present disclosure relates to dehydrocyclisation catalysts, methods of preparing dehydrocyclisation catalysts, and methods of dehydrocyclisation of hydrocarbons. These multi-functional catalysts are more stable (e.g., lower deactivation rates) than conventional catalysts for dehydrocyclisation and exhibit enhanced performance characteristics for aromatization of hydrocarbons (e.g., conversion, selectivity, yield, etc.). In general, the dehydrocyclisation catalysts include a suitable support and an organometallic complex or a coordination compound comprising, among other things, a dehydrogenation metal (and eventually a ligand). The dehydrogenation metal of the organometallic complex or coordination compound may be grafted to the suitable support at a selected site using, for example, Surface Organometallic Chemistry techniques. This ability to graft the dehydrogenation metal to a selected site of the support provides unprecedented control over the spatial distribution of active sites involved in the dehydrocyclisation of hydrocarbons and unprecedented stability by preventing coking inside the micropores of the support.

The dehydrocyclisation catalysts may be characterized by a spatial distribution and/or spatial separation of the dehydrocyclisation functions (e.g., dehydrogenation, oligomerisation, and cyclisation). The grafting techniques used to prepare the dehydrogenation catalysts provide control over the locations and/or spatial distribution of active sites involved in dehydrocyclisation of hydrocarbons. In general, the location includes the position of the chemical group on the support on which the complex is grafted (e.g., via the dehydrogenation metal). In preferred embodiments, the dehydrogenation metal is grafted to a site or surface spatially located outside or substantially outside the micropores of the support and the Brønsted sites for oligomerisation and cyclisation are located inside or substantially inside the micropores. Shape selectivity of the dehydrocyclisation catalyst comes from confinement of the Brønsted sites inside the micropores for cyclisation function. In other embodiments, the dehydrogenation metal may be grafted inside the micropores of the support. This controlled spatial separation prevents coking inside the micropores of the support, leading to much lower catalyst deactivation during aromatization of light alkanes.

The location of the active sites may further be controlled based on the size of the organometallic complex precursor or coordination compound precursor in relation to the pore structure of the support. Large organometallic complexes or coordination compounds with molecular diameters greater than the pore diameter of the support are generally only grafted on the outside surface of the support. For example, a large organometallic complex (e.g., TiNp₄, molecular diameter of about 1 nm) may be grafted on the outside surface of a ZSM-5 support because the precursor is precluded from entering the pores of the support, which are about 0.5 nm in diameter. Conversely, ZnMe₂, with a molecular diameter of about 0.4 nm, is grafted on the surface of the support and also inside the micropores. Accordingly, while the latter produces a catalyst with low stability (e.g., a relatively high deactivation rate), the former provides a catalyst with high stability and a low deactivation rate. This approach, either in addition or in the alternative to the grafting technique employed, permits separation of the different functions required for aromatization reaction of light alkanes.

The dehydrocyclisation catalyst may be used for the conversion of alkanes (e.g., light alkanes) to aromatics, such as benzene, toluene, and xylenes. The spatial separation of dehydrocyclisation function prevents coking inside the micropores of the support. For example, in many embodiments, the active site for the dehydrogenation function is outside the micropores of the support (e.g., at the metal site) and the active sites for the oligomerisation and cyclisation functions are located inside the micropores of the support, where the Brönsted acid sites may be naturally present as in the case of supports based on aluminosilicate frameworks. By spatially separating and/or distributing the dehydrocyclisation functions, the dehydrocyclisation catalyst prevents and/or limits coking, leading to a highly stable catalyst during the aromatization of hydrocarbons and enhancing conversion of alkanes and yield and selectivity of aromatics.

### Definitions

The terms recited below have been defined as described below. All other terms and phrases in this disclosure shall be construed according to their ordinary meaning as understood by one of skill in the art.

As used herein, “calcinating” refers to thermal treatment of any substance to cause removal of a volatile fraction, thermal decomposition, phase transition, or combination thereof. In many embodiments, calcinating refers to a high-temperature thermal treatment of a support in the presence of a gas/vapor to induce one of the enumerated physiochemical changes. The gas/vapor may include any gas/vapor. In many embodiments, the gas/vapor includes one or more of air and oxygen.

As used herein, “contacting” refers to the act of touching, making contact, or of bringing to immediate or close proximity, including at the molecular level, for example, to bring about a physiological reaction, a chemical reaction, or a physical change, e.g., in a solution, in a reaction mixture, in vitro, or in vivo. Accordingly, treating, tumbling, vibrating, shaking, mixing, and applying are forms of contacting to bring two or more components together.

As used herein, “contacting” may, in addition or in the alternative, refer to, among other things, feeding, flowing, passing, injecting, introducing, and/or providing the fluid composition (e.g., a feed gas).

As used herein, “CVD” refers to chemical vapor deposition.

As used herein, “dehydrating” refers to the removal of hydrogen from a compound. In many embodiments, the compound is an organic compound, such as a hydrocarbon.

As used herein, “hierarchical molecular sieve” refers to a molecular sieve material possessing both narrow and large pores. For example, a hierarchical zeolite may contain two or more types of pore sizes where additional larger pores (mesopores) can overcome possible transport limitations of the smaller pores.

As used herein, “hydrocarbon” refers to any molecule or compound consisting of hydrogen and carbon. A hydrocarbon may include, but is not limited to, any alkane, alkene, and alkyne. The hydrocarbon may be saturated or unsaturated, linear or branched, cyclic or acyclic, aromatic or non-aromatic, and may include any number of carbon and hydrogen atoms.

As used herein, “reacting” refers to forming a chemical bond (e.g., covalent bond). In some embodiments, the bond may be formed via grafting and/or contacting. Grafting may include techniques such as Surface Organometallic Chemistry and/or chemical vapor deposition.

As used herein, “SOMC” refers to Surface Organometallic Chemistry.

Embodiments of the present disclosure describe a catalyst for dehydrocyclisation of hydrocarbons comprising a suitable support and an organometallic complex or coordination compound including at least a dehydrogenation metal. In these embodiments, the dehydrogenation metal of the organometallic complex or coordination compound may be grafted to a selected site of the suitable support.

The suitable support may generally include any molecular sieve material. The molecular sieve material may include a microporous material, mesoporous material, macroporous material, and/or hierarchical molecular sieve material. In many embodiments, the molecular sieve material includes one or more of a zeolite, clays, alumina, silica, porous silica, and mesoporous silica. For example, the molecular sieve material may include, but is not limited to, one or more of ZSM-5, Faujasite, USY, LTL, Ferrierite, Merlinoite, Mordenite, KCC-1 (e.g., KCC-1 modified by aluminum), SBA-15, MCM-41, etc. The zeolite may include any zeolite known in the art, including, but not limited to, zeolites with any of the framework types approved by the Structure Commission of the International Zeolite Association (IZA-SC). In many embodiments, the zeolite includes one or more of: ZSM-5, Faujasite, USY, LTL, Ferrierite, Merlinoite, and Mordenite. From the total pore volume of the molecular sieve at least 40 vol % are micropores, but this is preferably higher than 60%. Micropores may have pore sizes ranging from about 0.3 nm to about 2 nm, preferably from about 0.3 nm to about 0.7 nm. The molecular sieve should also have at least about 0.05 mmol/g to about 1.5 mmol/g of Brönsted Acid Sites. In other embodiments, the molecular sieve material may include hierarchical molecular sieve material. For example, the hierarchical molecular sieve material may include mesoporous ZSM-5.

The organometallic complex or coordination compound generally includes a metal and a ligand. The metal of the organometallic complex or coordination compound includes one or more metals suitable for the dehydrogenation function of the dehydrocyclisation of hydrocarbons. Dehydrogenation metal site have preferably Lewis acidity able to accept electron pair and thus to make hydrides by abstraction from a hydrocarbon. These metals may be referred to as dehydrogenation metals. The dehydrogenation metals may include, among other things, alkali metals, alkaline earth metals, transition metals, post-transition metals, and lanthanides. For example, the dehydrogenation metal may include one or more of Ti, Zn, Ni, Mo, Nb, Ga, Sr, Sn, Cu, Ru, W, Ta, Cr, V, Hf, Co, Ce, Ir, Pt, Os, Zr, Cs, Li, Mg, Mn, Fe, V, Re, Pd, Au, Cd, Ag, Bi, and La. In many embodiments, the dehydrogenation metal includes Ti. The preferred dehydrogenation metals are Ti, Zn, Ga, W, Ta, Fe, Mo and Ni. The ligand of the organometallic complex or coordination compound may include one or more ligands suitable for forming a complex with the dehydrogenation metal. For example, the ligand may include any alkyl possibly associated with another fragment such as halides, alkoxides, and aryloxides, among others. In many embodiments, the organometallic complex or coordination compound includes one or more methyl, ethyl, n-propyl, tertiobutyl, isobutyl, isopentyl, neopentyl, cyclopentadienyl, phenyl ligands, and halogens (e.g., F, Cl, Br, and I), oxo, and alkoxo. In many embodiments, the metal may be bonded to the support through a covalent bond such that the organometallic complex is prevented from entering (e.g., cannot enter) the internal cavities and/or pores of the molecular sieve. In other embodiments, the size of the organometallic complex is greater than the size of the micropores in the molecular sieve to prevent the complex from entering the pores. The ligands may also be large. The organometallic complex or coordination compound may have a molecular diameter ranging from about 0.2 to about 1.5 nm. In a preferred embodiment, the molecular diameter ranges from about 0.4 nm to about 1.2 nm. In some embodiments, the organometallic complex or coordination compound is tetrakis(neopentyl) titanium, TiNp₄. In some embodiments, the ligands include one or more of tertiobutyl, cyclopentadienyl, isobutyl, phenyl, and benzyl, among others.

The dehydrogenation metal of the organometallic complex or coordination compound is grafted to a selected site of the suitable support. The selected site may be inside or substantially inside and/or outside or substantially outside the micropores of the support. In many embodiments, the selected site is a location (e.g., a surface) outside the micropores of the suitable support. In these embodiments, the location where the dehydrogenation metal is grafted to the selected site—which may also be referred to as the metal site—is the active site for dehydrogenation. The selected site is preferably a silanol site of a zeolite molecular sieve. In other embodiments, the selected site may be Si—O—Si resulting from the dihydroxylation of silica leading to a highly dehydroxylated surface. Further, in these embodiments, the active sites for oligomerisation and cyclisation are inside the micropores of the suitable support, where Brønsted acids are generally naturally present, for example, in the case of molecular sieves with aluminosilicate frameworks. Accordingly, the active sites for dehydrogenation are the metal sites and the active sites for oligomerization and cyclisation are the Brønsted sites located inside the micropores of the support. In other embodiments, the metal site is also an active site for metathesis reactions, leading to the required oligomers. In this way, the dehydrocyclisation catalyst includes a spatial distribution and/or spatial separation of active sites involved in the dehydrocyclisation of hydrocarbons.

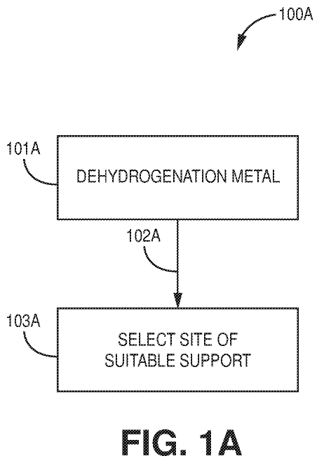

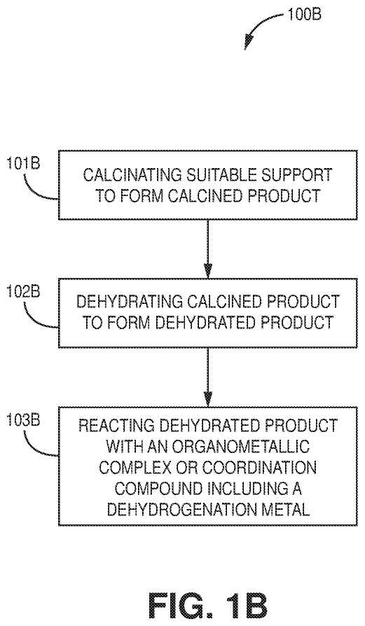

FIG. 1A is a flowchart of a method 100A of preparing a dehydrocyclisation catalyst for hydrocarbons, according to one or more embodiments of the present disclosure. As shown in FIG. 1A, a dehydrogenation metal of an organometallic complex or coordination compound 101A is grafted 102A to a selected site 103A of a suitable support to form a dehydrocyclisation catalyst. The dehydrocyclisation catalysts prepared according to the present method may include any of the organometallic complexes, coordination compounds, dehydrogenation metals, ligands, and suitable supports described above and elsewhere herein.

Grafting generally refers to techniques for creating chemical bonds between a metal and a support. In a preferred embodiment, the grafting technique is Surface Organometallic Chemistry. In another preferred embodiment, the grafting technique is chemical vapor deposition. In other embodiments, any grafting technique capable of creating a chemical bond between any of the dehydrogenation metals described herein and any of the suitable supports described herein may be used to prepare the dehydrocyclisation catalysts of the present disclosure, provided that the grafting technique provides the requisite control over the location of the selected site (e.g., the site where the dehydrogenation metal is grafted to the suitable support). In many embodiments, the chemical bond between the dehydrogenation metal and the suitable support is formed at the selected site. The selected site may include a surface of the suitable support, such as a surface spatially located outside or substantially outside the micropores of the suitable support. A non-limiting specific example is a dehydrogenation metal grafted to a silanol site of the suitable support via SOMC. The grafting site of the support may also be a Si—O—Si or Si—O—Al bond.

As described here and elsewhere, the grafting technique, among other things, may be selected to control and/or define the location of the active sites for dehydrogenation, oligomerization, and/or cyclisation. That is, in many embodiments, only grafting techniques that produce dehydrocyclisation catalysts with a spatial separation and/or spatial distribution of dehydrocyclisation functions (e.g., dehydrogenation, oligomerization, cyclisation, etc.) are selected. In preferred embodiments, SOMC is the grafting technique used to graft the dehydrogenation metal to the selected site (e.g., silanol site) because the SOMC technique provides control over the location where the dehydrogenation metal is grafted to the suitable support. For example, the SOMC technique may be used to graft a dehydrogenation metal to a silanol site of the suitable support.

In addition or in the alternative, relative diameters (e.g., diameters of the organometallic complex or coordination compound and micropores) may be selected to control and/or define the location of the active sites for dehydrogenation, oligomerization, and/or cyclisation. For example, selecting an organometallic complex with a diameter about greater than the pore diameter of the suitable support may further or independently provide control over the location where the dehydrogenation metal is grafted to the suitable support. Selection of dehydrogenation metals and/or suitable supports in this way may provide control over grafting of the dehydrogenation metal outside or substantially outside the micropores. These examples shall not be limiting. Any of the other grafting techniques and/or factors for controlling/defining the selected site described herein may be used.

In some embodiments, the SOMC technique is used to graft the dehydrogenation metal to the selected site of the suitable support. In these embodiments, grafting may include one or more steps of calcinating, dehydrating, and reacting. For example, FIG. 1B is a flowchart of a method 100B of preparing a dehydrocyclisation catalyst for hydrocarbons, according to one or more embodiments of the present disclosure. As shown in FIG. 1B, a dehydrocyclisation catalyst is prepared by one or more of calcinating 101B a suitable support to form a calcined product, dehydrating 102B the calcined product to form a dehydrated product, and reacting 103B the dehydrated product with an organometallic complex or coordination compound including a dehydrogenation metal. In some embodiments, the calcinating and dehydrating steps include pre-treatment steps and the reacting step includes grafting. The dehydrocyclisation catalyst may include any of the suitable supports, organometallic complexes, coordination compounds, dehydrogenation metals, and ligands described herein.

At step 101B, the suitable support is calcined to form a calcined product. Calcinating may include heating the suitable support to a calcination temperature in the presence of a gas for a period of time (e.g., calcination period) in order to, for example, remove water (e.g., surface water) and/or expose hydroxyl groups. The calcination may also occur under vacuum. The calcination temperature may range from about 300° C. to about 700° C. The duration of calcination may range from about 2 hours to about 16 hours. In many embodiments, calcinating includes exposing and/or heating the suitable support to a temperature ranging from about 400° C. to about 550° C. in the presence of air and/or oxygen for several hours. In a preferred embodiment, the temperature is about 550° C. and/or the duration of heating is at least about 5 hours. Calcinating the suitable support forms the calcined product, which has the Brønsted acidity.

At step 102B, the calcined product (e.g., the calcined suitable support) is dehydrated to form a dehydrated product. Dehydrating may include exposing and/or heating the calcined product to a dehydration temperature and dehydration pressure for a period of time (e.g., dehydration period) sufficient to remove water. The dehydration temperature may range from about 25° C. to about 300° C. The dehydration pressure may range from about 1×10⁻¹ mbar to about 1×10⁻² mbar. The duration of dehydration may range from about 10 hours to about 15 hours. In many embodiments, dehydrating includes exposing and/or heating the calcined product to a temperature ranging from about 25° C. to about 300° C. under vacuum for several hours. In a preferred embodiment, dehydrating includes exposing and/or heating the calcined product to about 300° C. for about 12 hours. Dehydrating the calcined product forms the dehydrated product.

At step 103B, the dehydrated product (e.g., the calcined and dehydrated suitable support) is reacted with an organometallic complex or coordination compound including a dehydrogenation metal to form the dehydrocyclisation catalyst. Reacting may include contacting the dehydrated product with the organometallic complex or coordination compound in a solvent at a reaction temperature for a reaction period sufficient to form the dehydrocyclisation catalyst. The solvent may include an organic solvent. For example, the solvent may include one or more of pentane, hexane, petroleum ether, THF, acetonitrile, and DMSO, among others. The reaction temperature may range from about −70° C. to about 150° C. The reaction period may range from about 0.5 hours to about 60 hours. In many embodiments, reacting includes contacting the dehydrated product and the organometallic complex or coordination compound in an organic solvent at a temperature ranging from about 25° C. to about 100° C. for at least about 5 hours. In a preferred embodiment, reacting includes contacting the dehydrated product and the organometallic complex or coordination compound in pentane at room temperature for about 2 days. In some cases, the grafting temperature may be below 25° C. (e.g., −100° C.).

An optional step 104B (not shown) includes one or more washings of the dehydrocyclisation catalyst with an organic solvent, such as pentane. An additional optional step 105B (not shown) includes drying (e.g., exposing and/or heating) the dehydrocyclisation catalyst under vacuum to remove solvent. For example, in embodiments in which pentane is used, drying may include heating under vacuum at about 80° C. for about 12 hours.

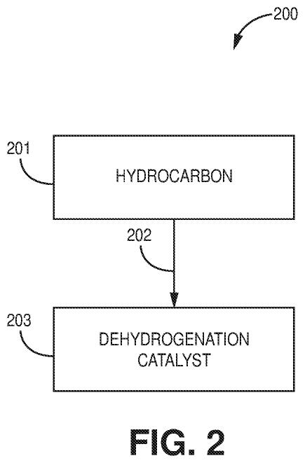

FIG. 2 is a flowchart of a method 200 of dehydrocyclisation of hydrocarbons, according to one or more embodiments of the present disclosure. As shown in FIG. 2, a hydrocarbon-containing feed stream 201 is contacted 202 with a dehydrocyclisation catalyst 203 to convert one or more of the hydrocarbons to an aromatic compound. In many embodiments, the dehydrocyclisation catalyst includes a dehydrogenation metal grafted to a selected site of a suitable support.

The hydrocarbon-containing feed stream 201 may include one or more alkanes (e.g., light alkanes) and/or other chemical species, each of which may exist in any phase (e.g., gas/vapor, liquid, solid). In general, the alkane may include any alkane. The alkane may be linear or branched, saturated or unsaturated, and/or cyclic or acyclic. In many embodiments, the alkane includes any C₁ to C₇ linear or branched alkane. For example, the alkane may include one or more of ethane, propane, n-butane, isobutane, n-pentane, 2-methylbutane, 2,2-dimethypentane. In other embodiments, the alkane includes one or more of ethane, propane, butane, pentane, isopentane, isobutene. The other chemical species may include other alkanes and hydrocarbons (e.g., C₅₊ hydrocarbons, olefins, etc.), nitrogen, oxygen, carbon dioxide, and/or other non-hydrocarbon species. The hydrocarbon-containing feed stream may be or be derived from, for example, natural gas, liquefied petroleum gas, and/or refinery/petrochemical streams, including waste streams. In many embodiments, methane is not included in the hydrocarbon-containing feed stream. In many embodiments, the hydrocarbon-containing feed stream may include between about 40 vol % to about 100 vol % of hydrocarbons.

The dehydrocyclisation catalyst 203 may include any of the catalysts described herein. In many embodiments, the dehydrocyclisation catalyst includes a dehydrogenation metal grafted via SOMC to a silanol site (or any reactive Si—O—Si or Si—O—Al) of a suitable support. The dehydrocyclisation catalysts may be characterized by spatial distribution and/or spatial separation of dehydrocyclisation functions, including, but not limited to, one or more of hydrogenation, oligomerisation, and cyclisation. For example, in many embodiments, hydrogenation occurs at the metal site (e.g., where the dehydrogenation metal is grafted to the silanol site), and oligomerisation and cyclisation occur at Brønsted sites located inside micropores of the molecular sieve. In some embodiments, the metal site is also active for metathesis. The shape selectivity of the dehydrocyclisation catalyst due to confinement of the Brønsted sites inside the micropores of the support favor cyclisation. In addition, the spatial separation of the dehydrocyclisation functions reduces and/or prevents coking inside the micropores of the support and thereby improves the stability of the catalyst over conventional catalysts.

Contacting may include feeding, flowing, passing, injecting, introducing, and/or providing a fluid composition, such as a feed stream. The contacting may occur at various pressures, temperatures, and concentrations of chemical species in the fluid composition, depending on desired feed conditions and/or reaction conditions. The pressure, temperature, and concentration at which the contacting occurred may be varied and/or adjusted according to a specific application. In many embodiments, the temperature ranges from about 350° C. to about 750° C. In preferred embodiments, the temperature ranges from about 400° C. to about 600° C. In many embodiments, the pressure ranges from about 0.5 atm to about 5 atm. In preferred embodiments, the pressure ranges from about 0.5 atm to about 2 atm. In one embodiment, the temperature for reaction is about 550° C., the pressure is about 1 bar, the duration of reaction is about two to three days, the concentration of propane in the feed is about 100%, and the feed of propane is about 20 ml/min)

The aromatic compounds may include one or more of benzene, toluene, and xylenes. In some embodiments, the method may further form other compounds. The compounds formed may include any hydrocarbon. For example, the compounds may include one or more of methane, ethane, ethylene, propylene, n-butane, iso-butane, trans-butene, cis-butene, toluene, benzene, and para-xylene. Other compounds may be formed based on the composition of the feed stream and these examples shall not be limiting.

The conversion of hydrocarbons to aromatic compounds according to the present disclosure may exhibit enhanced performance characteristics. In many embodiments, the enhanced performance characteristics may be defined by a value indicating a decrease of yield after about 2000 min TOS. The decrease in yield may be defined as follows: Yield Decrease=((Initial Yield−Yield after 2000 min)/Initial Yield)*100. In these embodiments, the yield decrease may range from about 30% to about 50%.

In other embodiments, the enhanced performance characteristics may be characterized by conversion, yield, and selectivity. In general, the conversion, yield, and selectivity is unprecedented and high. For example, the initial conversion (at time zero) of alkane to aromatic compounds may range from about 30% to about 100%. In many embodiments, the conversion ranges from about 40% to about 100%. In preferred embodiments, the conversion ranges from about 50% to about 100%. The conversion after 2000 min time-on-stream of alkane to aromatic compounds may range from about 20% to about 70%. In many embodiments, the conversion after 2000 min ranges from about 25% to about 45%. In preferred embodiments, the conversion after 2000 min ranges from about 30% to about 40%. The initial yield of aromatic compounds may range from about 15% to about 65%. In many embodiments, the yield of aromatic compounds may range from about 20% to about 50%. In preferred embodiments, the yield of aromatic compounds may range from about 25% to about 45%. The yield of aromatic compounds (BTX) after 2000 min time-on-stream may range from about 5% to about 55%. In many embodiments, the yield of aromatic compounds after 2000 min may range from about 10% to about 45%. In preferred embodiments, the yield of aromatic compounds after 2000 min may range from about 10% to about 40%. The selectivity of aromatic compounds and non-aromatic compounds ranges from about 40% to about 90%. In many embodiments, the selectivity of aromatic compounds to non-aromatic compounds ranges from about 45 to about 80%. In preferred embodiments, the selectivity of aromatic compounds to non-aromatic compounds ranges from about 50% to about 70%.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

### Synthesis of TiNp₄ Grafted on ZSM-5 by SOMC Approach

The zeolite H-ZSM-5 form (Parent-ZSM-5) was obtained after calcination of commercial-ZSM-5 (ammonium form) at 550° C. under air during 5 h. After calcination, the product was dehydrated under vacuum (10-mbar) at 300° C. for 12 h named Parent-ZSM-5₋₃₀₀. The Parent-ZSM-5₋₃₀₀ reacted with 1.2 equivalent (0.6 mmol) of TiNp₄ at room temperature in pentane for 2 days. The grafted titanium complex maned [≡Si—O—TiNp₃] was obtained after repeated washings with pentane followed by removing the solvent under vacuum at 80° C. for 12 h. Scheme 1 illustrates the synthesis of supported titanium complex [≡Si—O—TiNp₃]

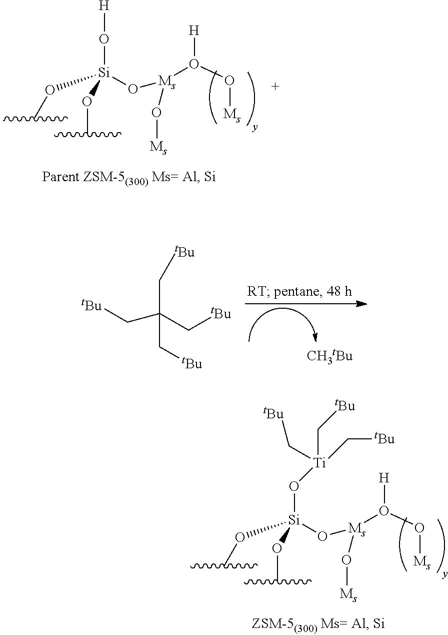

### Example 2

### Synthesis of ZnMe Grafted on ZSM-5 by SOMC Approach

0.8 mmol of ZnMe₂ was reacted with 1 g of Parent-ZSM-5, dehydrated at 300° C., under argon at room temperature for 24 h. The final product ZnMe@Parent ZSM-5 was obtained after repeated washings with pentane followed by evaporation of solvent under vacuum at 80° C. for 12 h. Scheme 2 illustrates the synthesis of supported [≡Si—O—ZnMe].

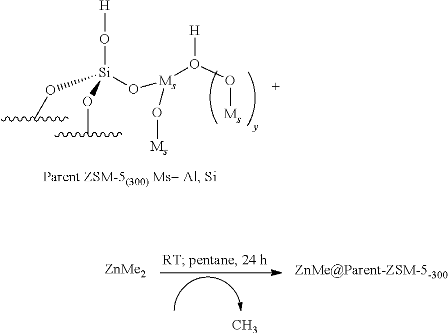

The elemental analysis of Zn shows the presence of 2.7 wt. % of Zn grafted on the support, which correspond to 0.4 mmol of Zn grafted. The solid state NMR for this simple was done by ¹H and ¹³C NMR, the spectrum shows the presence of one signal at 0 ppm on ¹H NMR. The ¹³C CP/MAS NMR shows the presence of two signals at 0 and −20 ppm corresponding to Si—CH₃ and [≡Si—O—ZnMe], respectively.

### Example 3

### Synthesis of Ga Grafted on ZSM-5 by Incipient Wetness Impregnation

A solution of 0.7 M of Ga(NO₃)₃.9H₂O was added to 1 g of ZSM-5 using a the high-throughput Chemspeed apparatus. This apparatus is an automated workstation allowing liquid and solids handling, shaking, cooling/heating and evaporation of several samples. After preparation, this catalysis was calcined at 550° C. for 6 h under air. The catalyst was characterised by elementary analysis of Ga and by N₂ adsorption/desorption. The elemental analysis for Ga shows the presence of 1 wt. % of Ga as expected. The N₂ adsorption desorption shows the BET surface for catalyst after grafting decrease slightly for 528 to 520 m²·g⁻¹.

### Example 4

### Synthesis of Ga Grafted on ZSM-5 by SOMC Approach

The zeolite H-ZSM-5 form (Parent-ZSM-5) was obtained after calcination of commercial-ZSM-5 (ammonium form) at 550° C. under air during 5 h. After calcination, the product was dehydrated under vacuum (10⁻⁵ mbar) at 300° C. for 12 h named Parent-ZSM-5₋₃₀₀. The Parent-ZSM-5₋₃₀₀ reacted with 1.2 equivalent (0.5 mmol) of Ga(ⁱBu)₃ at room temperature in pentane for 24 hours. The grafted titanium complex maned [(≡Si—O—)Ga(ⁱBu)₂] was obtained after repeated washings with pentane followed by drying of solvent under vacuum at 80° C. for 12 h. Scheme 1 illustrates the synthesis of supported titanium complex [(≡Si—O—)Ga(ⁱBu)₂].

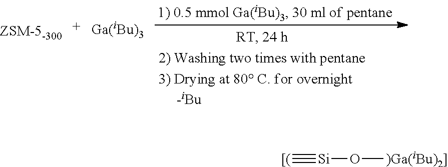

### Example 5

### Catalytic Activity of Different Catalysis (Parent-ZSM-5, [≡Si—O—ZnCH₃], [≡Si—O—TiNp₃], [(≡Si—O—)Ga(ⁱBu)₂] and Ga@ZSM-5) on the Conversion of Propane at 550° C. for 12 h

The conversion of propane in presence of different catalysis Ti, Ga and Zn grafted on ZSM-5 as well as for ZSM-5 were performed at 550° C. for 12 h in PID reactor. During the reaction, various products was obtained including methane, ethane, ethylene, propylene, n-butane, iso-butane, trans-butene, cis-butene, toluene, benzene and para-xylene.

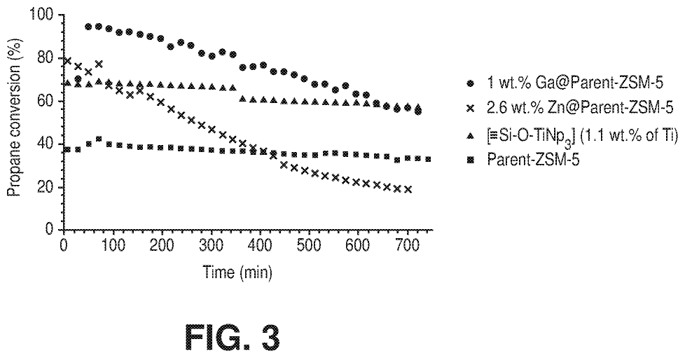

FIG. 3 shows the conversion of propane decrease very fast in presence of Ga@ZSM-5 and ZnMe@ZSM-5. However, in presence of TiNp@ZSM-5 and Parent ZSM-5, the conversion of propane are quite stable during the reaction. In comparison between TiNp@ZSM-5 and ZnMe prepared by SOMC approach shows a net difference of conversion of propane. Due to the grafted of TiNp on the surface of ZSM-5, allowed a good activity of this catalyst.

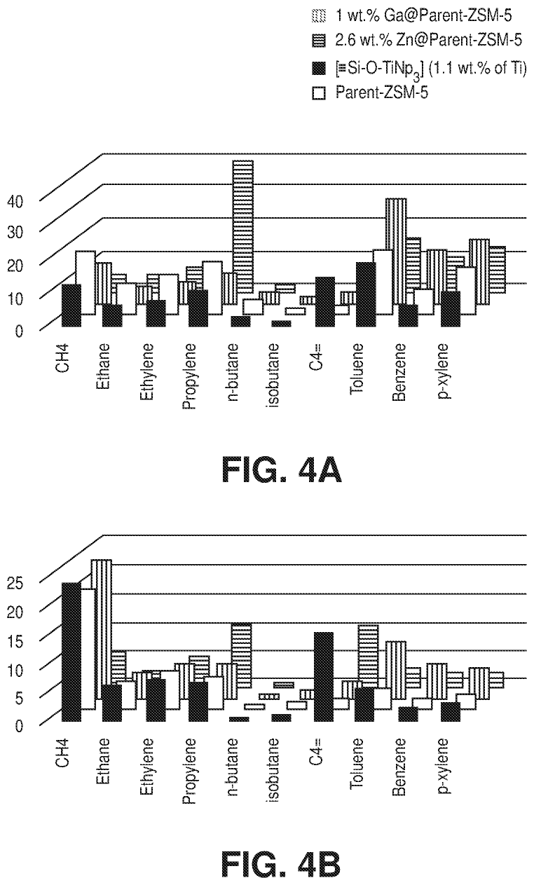

FIG. 4A shows the Ga@ZSM-5 and [≡Si—O—TiNp₃] catalyst products a selectivity of aromatics (BTX) around 35-40%, this selectivity is quite equal in presence of Parent-ZSM-5. The selectivity to aromatics in presence of [≡Si—O—ZnCH₃] is good but decreases very fast in favour of propylene.

The selectivity to propylene is quite stable in presence of [≡Si—O—TiNp₃], Ga@ZSM-5 and Parent-ZSM-5.

The average yield of aromatics is much better in presence of [≡Si—O—TiNp₃] and Ga@ZSM-5 (FIG. 4B). Vecan notice the high yield of C4 olefins (trans-2-butene, 1-butene, iso-butene and cis-2-butene) in presence of [≡Si—O—TiNp₃] and [≡Si—O—ZnCH₃] due to reaction of olefins metathesis occurring on the active site of Ti and Zn.

### Example 6

### Catalytic Activity of Parent-ZSM-5, [≡Si—O—TiNp₃] and Ga@ZSM-5) on the Conversion of Propane at 550° C. for Three Days

The conversion of propane in presence of [≡Si—O—ZnMe] was performed at 550° C. for 12 h in PID reactor. During the reaction, various products were obtained including methane, ethane, ethylene, propylene, n-butane, iso-butane, trans-butene, cis-butene, toluene, benzene and para-xylene.

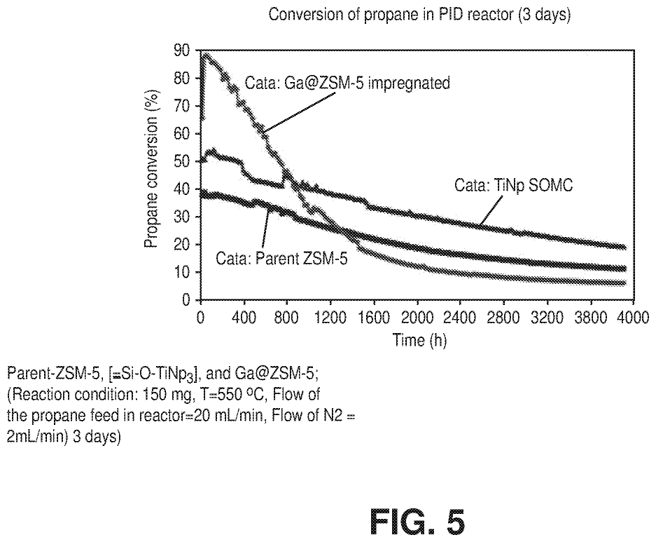

After this result obtained during 12 hours of reaction, the aromatization of propane reaction for more time (3 days) in presence of [≡Si—O—TiNp₃] was studied. The catalytic activity for this catalyst was compared to Parent-ZSM-5 and Ga@ZSM-5. The [≡Si—O—TiNp₃] catalyst shows a good and quite stable conversion of propane after 3 days compared to Ga@ZSM-5 (FIG. 5). The conversion of propane was around 90% in presence of Ga@ZSM-5 and decrease to 40% during the first day and become very low (5%) after 3 days t. The conversion of propane after three days is around 30% in presence of parent-ZSM-5.

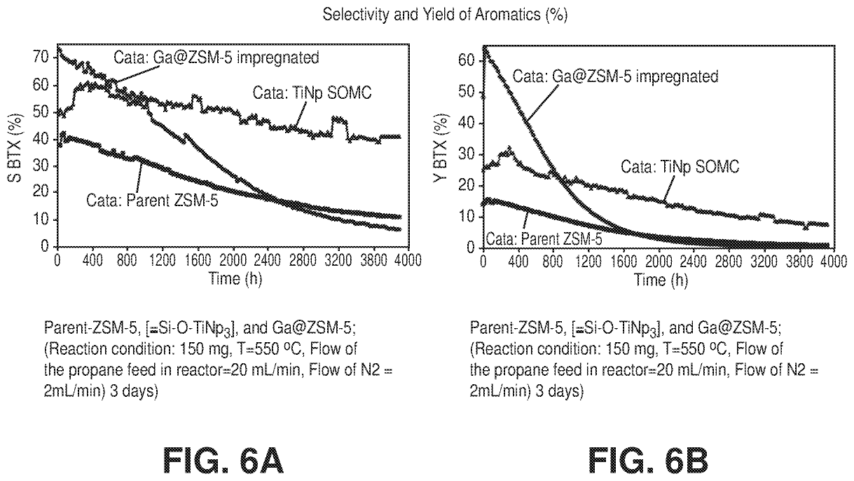

The selectivity to aromatics in presence of Ga@ZSM-5 is higher compared to [≡Si—O—TiNp₃] in the beginning of reaction, but it decreases very fast and became lower after 24 hours FIG. 6A.

FIG. 6B shows the yield of aromatics higher in presence of Ga@ZSM-5 and it decreases fast to 0% during the reaction. Conversely, the yield of aromatics is more stable in presence of [≡Si—O—TiNp₃].

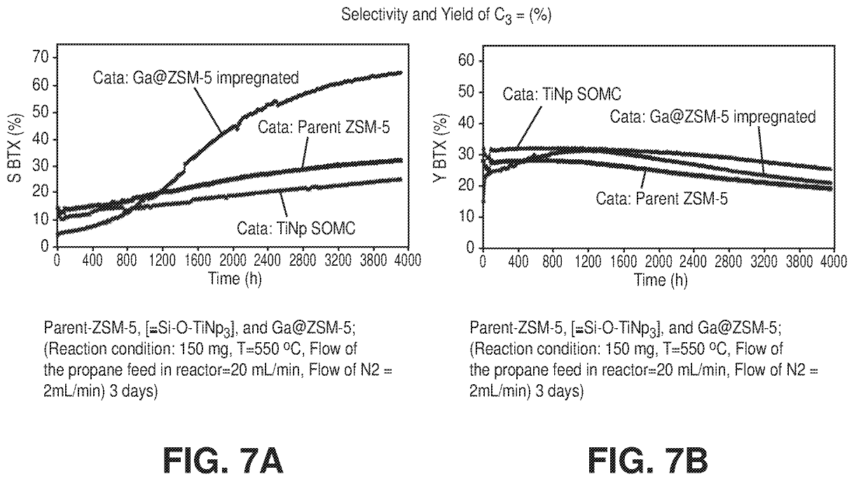

The selectivity of propylene (as an intermediate product) was also presented on this study, FIG. 7A shows that the selectivity of propylene increase very fast in presence of Ga@ZSM-5 toward the selectivity to aromatics as mentioned before. In presence of Ti catalyst, the selectivity to propylene is very low during the reaction.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with, or substituted for, one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A catalyst for dehydrocyclisation of hydrocarbons, comprising:
a suitable support, and
an organometallic complex or a coordination compound including at least a dehydrogenation metal, wherein the dehydrogenation metal of the organometallic complex or coordination compound is grafted to a selected site of the suitable support.

2. The catalyst of claim 1, wherein the suitable support includes a molecular sieve material.

3. The catalyst of claim 1, wherein the suitable support includes one or more of ZSM-5, USY, LTL, Mordenite, mesoporous ZSM-5, and KCC-1 modified by Aluminum.

4. The catalyst of claim 1, wherein the dehydrogenation metal includes one or more of Ti, Zn, Nb, Ga, Sr, Sn, Cu, Ru, W, Ta, Cr, V, Hf, Co, Ce, Ir, Pt, Os, Zr, Cs, Li, Mg, Mn, Fe, V, Re, Pd, Au, Cd, Ag, Bi, and La.

5. The catalyst of claim 1, wherein the organometallic or coordination complex further includes one or more methyl, ethyl, n-propyl, tertiobutyl, isobutyl, isopentyl, neopentyl, cyclopentadienyl, phenyl ligands, F, Cl, Br, I, oxo, and alkoxo.

6. The catalyst of claim 1, wherein the dehydrogenation metal is grafted to the selected site using one or more of Surface Organometallic Chemistry techniques and chemical vapor deposition techniques.

7. The catalyst of claim 1, wherein grafting the dehydrogenation metal to the selected site prevents coking.

8. The catalyst of claim 1, wherein the selected site is any site located spatially outside micropores of the suitable support.

9. The catalyst of claim 1, wherein the selected site is a silanol site.

10. The catalyst of claim 1, wherein a molecular diameter of the organometallic complex or coordination compound is greater than a pore diameter of the suitable support.

11. A method of preparing a dehydrocyclisation catalyst for hydrocarbons, comprising:
grafting a dehydrogenation metal of an organometallic complex or coordination compound to a selected site of a suitable support to form a dehydrocyclisation catalyst.

12. The method of claim 11, wherein grafting includes grafting using one or more of Surface Organometallic Chemistry techniques and chemical vapor deposition techniques.

13. The method of claim 11, wherein grafting includes one or more of:
calcinating the suitable support to form a calcined product;
dehydrating the calcined product to form a dehydrated product; and
reacting the dehydrated product with an organometallic complex or coordination compound.

14. A method of dehydrocyclisation of hydrocarbons, comprising:
contacting a hydrocarbon-containing feed stream with a dehydrocyclisation catalyst to convert one or more of the hydrocarbons to an aromatic compound, wherein the dehydrocyclisation catalyst includes a dehydrogenation metal grafted to a selected site of a suitable support.

15. The method of claim 14, wherein the hydrocarbon-containing feed stream includes one or more alkanes and/or one or more other chemical species.

16. The method of claim 14, wherein the hydrocarbon-containing feed stream includes one or more of ethane, propane, butane, and pentane.

17. The method of claim 14, wherein the aromatic compound includes one or more of benzene, toluene, and xylenes.

18. The method of claim 14, wherein a conversion of the aromatic compound ranges from about 10% to about 90%.

19. The method of claim 14, wherein a selectivity of the aromatic compound ranges from about 10% to about 70%.

20. The method of claim 14, wherein a yield of the aromatic compound ranges from about 10% to about 45%.
